(19) United States
(12) Patent Application Publication
(10) Pub. No.: US 2008/0177342 A1
(43) Pub. Date: Jul. 24, 2008

(54) Defibrillator With Impedance-Compensated Energy Delivery

(75) Inventor: David Snyder, Bainbridge Island, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/909,470

(22) PCT Filed: Mar. 23, 2006
(86) PCT No.: PCT/IB06/50905

§ 371 Date: Sep. 24, 2007

Related U.S. Application Data
(60) Provisional application No. 60/667,270, filed on Mar. 29, 2005.
(60) Provisional application No. 60/689,214, filed on Jun. 9, 2005.

(51) Int. Cl. A61N 1/00

(52) U.S. Cl. 607/8; 607/5; 607/6

(57) ABSTRACT

An external defibrillator is described which maintains the durations of phases of a multiphasic shock waveform within or below desired limits. As the duration of a waveform increases for patients of increased patient impedance, the durations of the phases of a multiphasic shock waveform also increase. Before a maximum duration limit is exceeded, the defibrillator adds another phase to the multiphasic waveform which brings the durations of the phases within the desired range or below a maximum duration limit. Both the number of shock phases and the individual phase durations can be controlled in response to measured patient impedance.

This invention relates to electrotherapy devices and methods and, in particular, to a defibrillator which provides for an impedance-compensated delivery of defibrillation pulses to a patient.

Sudden cardiac death is the leading cause of death in the United States. Most sudden cardiac death is caused by ventricular fibrillation, in which the heart's muscle fibers contract without coordination, thereby interrupting normal blood flow to the body. Electro-chemical activity within a human heart normally causes the heart muscle fibers to contract and relax in a synchronized manner that results in the effective pumping of blood from the ventricles to the body's vital organs. Sudden cardiac death is often caused by ventricular fibrillation (VF) in which abnormal electrical activity within the heart causes the individual muscle fibers to contract in an unsynchronized and chaotic way. The only effective treatment for VF is electrical defibrillation in which an electrical shock is applied to the heart to depolarize the myocardium and allow the heart's electro-chemical system to re-synchronize itself. Once organized electrical activity resumes, synchronized muscle contractions usually follow, leading to the restoration of cardiac rhythm.

To be effective, the defibrillation shock must be delivered to the patient within minutes of the onset of ventricular fibrillation. Studies have shown that defibrillation shocks delivered within one minute after ventricular fibrillation begins achieve up to 100% survival rate. The survival rate falls to approximately 30% if 6 minutes elapse before the shock is administered. Beyond 12 minutes, the survival rate approaches zero.

The minimum amount of patient current and energy delivered that is required for effective defibrillation depends upon the particular shape of the defibrillation waveform, including its amplitude, duration, shape (such as sine, damped sine, square, exponential decay), and whether the current waveform has a single polarity (monophasic), both negative and positive polarities (biphasic) or multiple negative and positive polarities (multiphasic). At the same time, there exists a maximum value of current in the defibrillation pulse delivered to the patient above which will result in damage to the myocardial tissue by electroporation and decreased efficacy of the defibrillation pulse. To prevent this, defibrillators often limit the peak current that occurs during delivery of the defibrillation pulse as discussed in U.S. Pat. No. 6,241,751 to Morgan et al.

Defibrillator waveforms, i.e., time plots of the delivered current or voltage pulses, are characterized according to the shape, polarity, duration and number of pulse phases. Most current defibrillators deliver monophasic current or voltage electrotherapeutic pulses, although some deliver biphasic sinusoidal pulses. Other prior art defibrillators use truncated exponential, biphasic waveforms. Examples of biphasic defibrillators may be found in U.S. Pat. No. 4,821,723 to Baker, Jr., et al.; U.S. Pat. No. 5,083,562 to de Coriolis et al.; U.S. Pat. No. 4,800,883 to Winstrom; U.S. Pat. No. 4,850,357 to Bach, Jr.; U.S. Pat. No. 4,953,551 to Mehra et al.; and U.S. Pat. No. 5,230,336 to Fain et al.

A defibrillator should deliver a waveform which is both effective for defibrillation and safe so as to prevent myocardial damage. An effective waveform will deliver a prescribed amount of energy, or dose, to the patient's heart. The amount of energy delivered to a patient for a given pulse will vary from patient to patient with the transthoracic impedance or patient impedance. Because the patient impedance of the human population may vary across a range spanning 20 to 200 ohms, it is desirable that a defibrillator provide an impedance-compensated defibrillation pulse that delivers a desired amount of energy to any patient with the range of patient impedances. The most prevalent way to control energy delivery across the range of patient impedances is by controlling the “tilt” or difference between initial and final voltages of the energy storage capacitor of the defibrillator as well as the discharge time of the defibrillation pulse. Most defibrillators use a single energy storage capacitor charged to a fixed voltage level resulting in a broad range of possible discharge times and tilt values across the range of patient impedances. A method of shaping the waveform of the defibrillation pulse in terms of duration and tilt is discussed in U.S. Pat. No. 5,607,454, “Electrotherapy Method and Apparatus” to Gliner et al.

Defibrillation output waveforms used by clinically available defibrillators are produced by capacitor discharge. Internal or implantable defibrillators, as well as some external or transthoracic defibrillators, utilize truncated exponential defibrillation waveforms. The waveforms are produced by charging the capacitors to a selected initial voltage and then allowing the capacitors to discharge for a period of time through defibrillation leads placed in or on the body so that current flows through the heart. The rate of capacitor discharge is dependent upon the impedance of the system including the patient impedance. These truncated exponential waveforms can be designed to have either “fixed tilt” or “fixed pulse width” as well as hybrid designs that try to strike a balance between the two. Fixed tilt defibrillators discharge the capacitors from the selected initial voltage until a predetermined final voltage is reached. This can be accomplished by either monitoring the voltage or by measuring the impedance, calculating the time required to reach the desired voltage, then controlling the waveform duration, the “tilt” being the percentage decline in capacitor voltage from its initial value; therefore, the pulse duration varies directly with the system impedance. In contrast, fixed pulse width defibrillators discharge their capacitors for a preselected duration and, as a result, the tilt of the waveform varies inversely with the impedance of the system; low impedances cause the waveform to have a high tilt, while high impedances result in low tilt.

One way of delivering rapid defibrillation shocks is through the use of implantable defibrillators. Implantable defibrillators are surgically implanted in patients who have a high likelihood of needing electrotherapy in the future. Implanted defibrillators typically monitor the patient's heart activity and automatically supply electrotherapeutic pulses directly to the patient's heart when indicated. Thus, implanted defibrillators permit the patient to function in a somewhat normal fashion away from the watchful eye of medical personnel. Implantable defibrillators are expensive, however, and are used on only a small fraction of the total population at risk for sudden cardiac death. Because each implanted defibrillator is dedicated to a single patient, its operating parameters, such as electrical pulse amplitudes and total energy delivered, may be effectively titrated to the physiology of the patient and to the patient impedance prior to implantation to optimize the defibrillator's effectiveness. Thus, for example, the initial voltage, first phase duration and total pulse duration may be set when the device is implanted to deliver the desired amount of energy or to achieve a desired start and end voltage differential (i.e., a constant tilt). Even when an implanted defibrillator has the ability to change its operating parameters to compensate for changes in the impedance of the defibrillators leads and/or the patient's heart (as discussed in the Fain patent), the range of potential impedance changes for a single implantation in a single patient is relatively small.

External defibrillators send electrical pulses to the patient's heart through electrodes applied to the patient's torso. External defibrillators are useful in the emergency room, the operating room, emergency medical vehicles or other situations where there may be an unanticipated need to provide electrotherapy to a patient on short notice. The advantage of external defibrillators is that they may be used on a patient as needed, then subsequently moved to be used with another patient. However, because external defibrillators deliver their electrotherapeutic pulses to the patient's heart indirectly (i.e., from the surface of the patient's skin rather than directly to the heart), they must operate at higher energies, voltages and/or currents than implanted defibrillators. Because external defibrillator electrodes are not in direct contact with the patient's heart, and because external defibrillators must be able to be used on a variety of patients having a variety of physiological differences, external defibrillators must operate according to pulse amplitude and duration parameters that will be effective in most patients, no matter what the patient's physiology. For example, the impedance presented by the tissue between external defibrillator electrodes and the patient's heart varies from patient to patient, thereby varying the intensity and waveform shape of the shock actually delivered to the patient's heart for a given initial pulse amplitude and duration. Pulse amplitudes and durations effective to treat low impedance patients do not necessarily deliver effective and energy efficient treatments to high impedance patients, and vice versa.

Since the variety of waveform parameters that may be controlled is substantial, various measures have been proposed for responding to differences in patient impedance. An effective dose can be measured by the amount of energy delivered to the patient which, for a given capacitance, is indicated by the decline in capacitor voltage from the time of pulse initiation to the time of pulse termination for a given tilt. Hence, the duration of the pulse is a variable that has been adjusted in response to patient impedance. The Fain et al. patent referenced above describes a defibrillator which automatically adjusts the pulse duration based upon the impedance measured or calculated following a delivered shock. U.S. Pat. No. 5,607,454 to Carmon et al. describe a defibrillator which automatically adjusts the pulse duration based upon an impedance measured as the shock is delivered. For multi-phasic systems the widths of each phase in the sequence can be controlled. Generally pulse widths are chosen so that the waveform will have a relatively constant tilt over a wide range of impedances for a given source capacitance. The widths of each phase can be kept equal or can be unequal for positive and negative phase durations with the width ratio kept constant or varied. Different capacitances of a capacitive network can be chosen and used in response to patient impedance as described in the Morgan et al. patent. U.S. Pat. No. 5,999,852 to Elabbady et al. describes a defibrillator which responds to measured patient impedance by controlling the phase duration of the pulse and the voltage level to which the capacitor of the defibrillator is charged. U.S. Pat. No. 6,738,664 to McDaniel advocates the proposition that it is better to optimize the frequency of stimulation rather than the appearance of the waveform in the time domain. The techniques described in this patent maintain a 100 Hz stimulation frequency while maintaining a constant tilt of 75% and constant phase durations; the overall duration of the shock varies correspondingly. There remains however the need to more efficiently deliver a therapeutically effective dose for patients over the full range of patient impedances. It is further desirable to deliver a pulse at the lowest output level that will result in a desired high probability of defibrillation to minimize the detrimental effects of delivering excessive energy to the heart. Attaining this further desire will also avoid depleting battery energy by the delivery of therapeutically ineffective pulse energy.

In accordance with the principles of the present invention, a defibrillator and electrotherapeutic method are described which improves the efficiency of therapeutically effective dose delivery for defibrillation. In one embodiment a method or apparatus of the present invention varies the number of phases of a defibrillation pulse in relation to a patient parameter such as patient impedance. For patients of increasing impedance, for example, the number of phases of the defibrillation waveform is increased. In accordance with another aspect of the present invention the durations of the phases of the defibrillation waveform are controlled in response to the patient parameter. As the waveform duration demands of a high impedance patient call for phase durations in excess of certain maximum or range values, the defibrillator responds by increasing the number of pulse phases with corresponding decrease in phase duration. An embodiment of the present invention may be configured to achieve a constant tilt. An embodiment of the present invention may employ phases of different durations.

In the drawings:

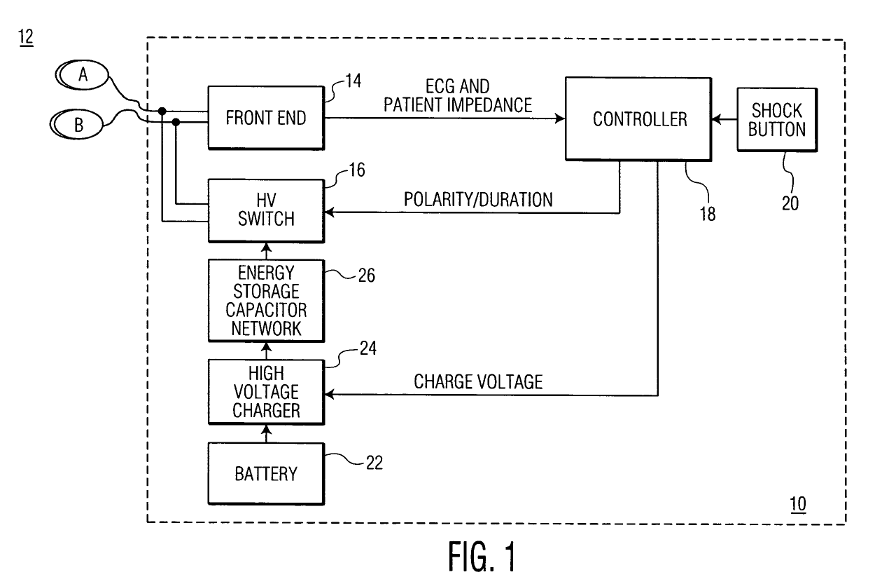

FIG. 1 illustrates in block diagram form a defibrillator which controls an output waveform in accordance with the principles of the present invention.

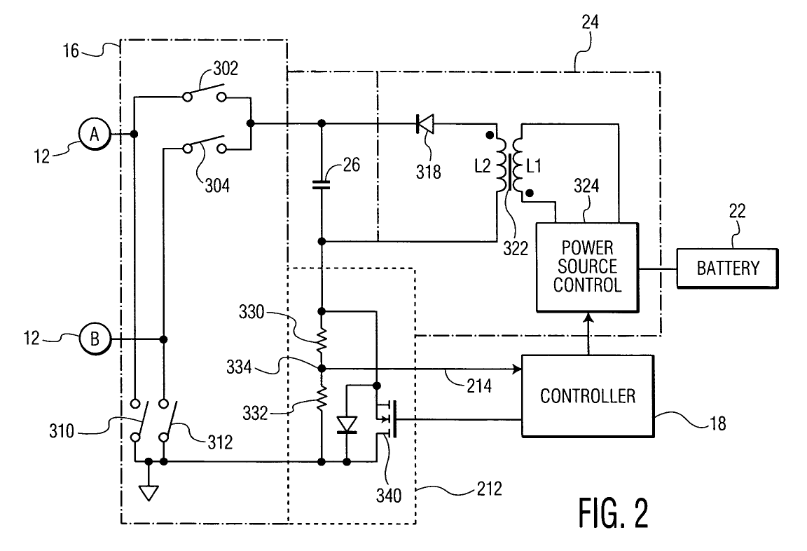

FIG. 2 illustrates the control and high voltage section of a defibrillator in schematic detail.

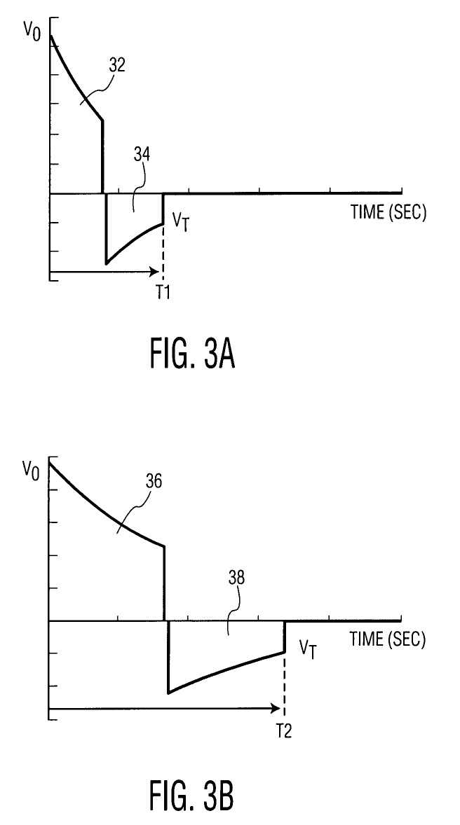

FIGS. 3A and 3B illustrate biphasic waveforms for dose delivery to low and high impedance patients.

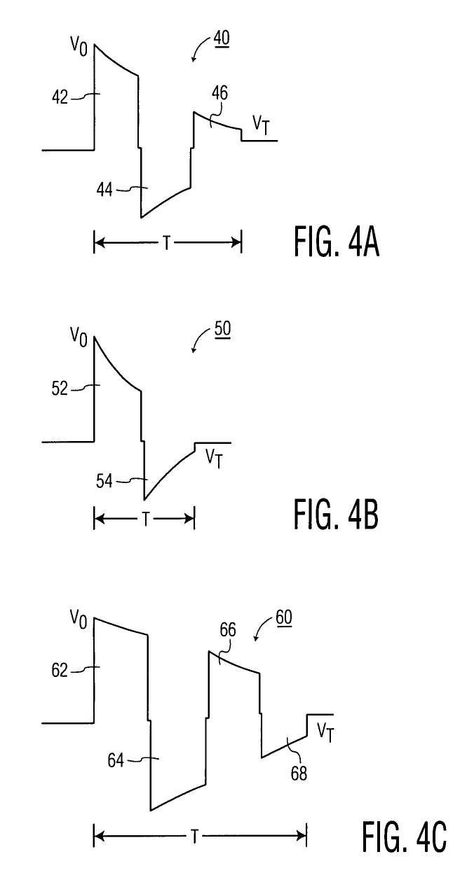

FIGS. 4A-4C illustrate defibrillation waveforms formed in accordance with the principles of the present invention.

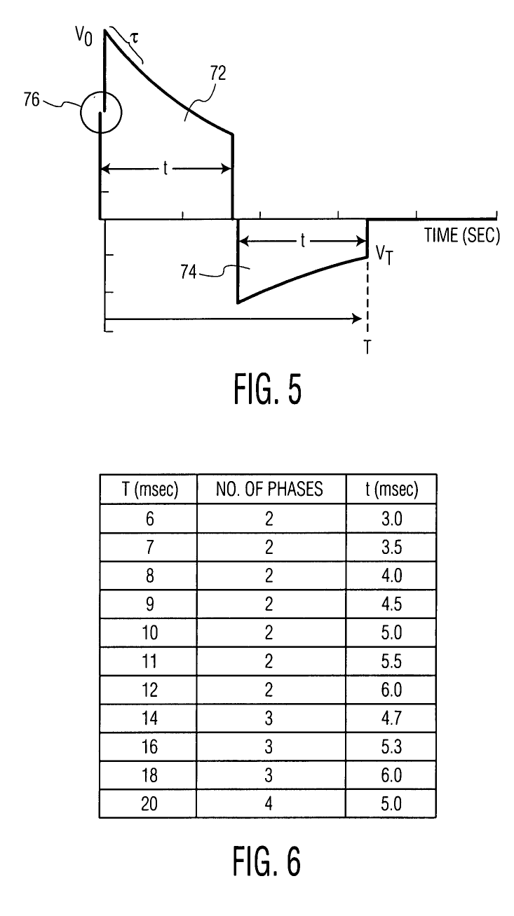

FIG. 5 illustrates techniques for measuring patient impedance.

FIG. 6 is a table of waveform characteristics for delivering waveforms in accordance with the principles of the present invention.

Referring first to FIG. 1, a simplified block diagram of a defibrillator 10 according to the present invention is shown. A pair of electrodes 12A & B for coupling to a patient (not shown) are connected to a front end 14 and further connected to a high voltage (HV) switch 16. The front end 14 provides for detection, filtering, and digitizing of the ECG signal and patient impedance from the patient. The ECG signal is in turn provided to a controller 18 which runs a shock advisory algorithm that is capable of detecting ventricular fibrillation (VF) or other shockable rhythm that is susceptible to treatment by electrotherapy.

The front end 14 is capable of measuring the patient impedance across the electrodes 12 by any one of several techniques described below. One such technique is applying and measuring the response of the patient to a low level test signal. By this technique a low-level non-therapeutic electrical signal is delivered to the patient prior to delivery of the defibrillation pulse and the voltage induced across the electrodes 12 in response thereto is measured. The patient impedance is measured and digitized in the front end 14 using an analog to digital converter (not shown) in order to provide the patient impedance data to the controller 18.

A shock button 20, typically part of a user interface of the defibrillator 10, allows the user to initiate the delivery of a defibrillation pulse through the electrodes 12 after the controller 18 has detected VF or other shockable rhythm. A battery 22 provides power for the defibrillator 10 in general and in particular for a high voltage charger 24 which charges the capacitors in an energy storage capacitor network 26. Typical battery voltages are 12 volts or less, while the capacitors in the energy storage capacitor network 26 may be charged to 1500 volts or more. A charge voltage control signal from the controller 18 determines the charge voltage on each capacitor in the energy storage capacitor network 26.

The energy storage capacitor network 26 contains one or multiple capacitors which may be arranged in series, parallel, or a combination of series and parallel arrangements responsive to a configuration control signal from the controller 18. The energy storage capacitor network 26 has an effective capacitance and effective charge voltage that depend on the selected configuration. For example, a configuration that consists of three series capacitors with a capacitance value C and charge voltage V will have an effective capacitance of 1/3 C and effective voltage of 3 V. Various suitable configurations are described in the aforementioned '751 patent to Morgan et al. The controller 18 uses the patient impedance and the dose energy level to select a configuration of the energy storage capacitor network 26 from the set of configurations in order to deliver the impedance-compensated defibrillation pulse to the patient.

The energy storage capacitor network 26 is connected to the HV switch 16 which operates to deliver the defibrillation pulse across the pair of electrodes 12 to the patient in the desired polarity and duration, in response to a polarity/duration control signal from the controller 18. The HV switch 16 is constructed using an H bridge to deliver multiphasic defibrillation pulses in the illustrated embodiment but could readily be adapted to deliver monophasic pulses if desired.

A defibrillator energy delivery system suitable for use in an embodiment of the present invention is shown in FIG. 2. The HV energy circuit 24 includes a transformer 322 with a primary coil LI connected to a power source control circuit 324. The power source control circuit 324 is connected to the battery 22, which serves as a source of DC current. The power source control circuit 324 can be any well known power switch circuitry now or later developed that provides an alternating current across the primary coil L1 of the transformer 322. Typically, the power source control circuit includes a field-effect transistor (FET) switch (not shown) connected to ground that applies a current pulse to the primary coil L1 of the transformer 322. The switch is controlled by the controller 18 to cause either an alternating current or a constant current across the primary coil L1. A diode 318 coupled to a secondary coil L2 of the transformer 322 rectifies the alternating current generated at the secondary coil L2, resulting in a series of positive current pulses being generated by the HV energy circuit 24. The charge capacitor 26 is coupled across the output of the HV energy circuit 24 to be charged in preparation for defibrillation. The charge delivery switch 16 connects the charge capacitor 26 to electrodes 12A and 12B in response to one or more shock control signals generated by the controller 18 in response to the shock button 20. In the embodiment illustrated in FIG. 2 the charge delivery switch 16 is implemented as an H-bridge electrically coupling the charge capacitor 26 to electrodes 12A and 12B.

In alternative embodiments of the present invention, alternative designs for the charge delivery switch 16 can be used. The H-bridge in the illustrated embodiment includes switches 302, 304, 310 and 312 to control the electrical connection between the charge capacitor 26 and the electrodes 12A and 12B. It should be understood that the H-bridge of the charge delivery switch 16 can be controlled to apply, for example, monophasic or biphasic defibrillation pulses to the electrodes 12.

The energy delivered to the patient from the capacitor 26 by the charge delivery switch 16 can be monitored or measured by a measurement circuit 212. The measurement circuit 212 includes a pair of series coupled resistors 330, 332, and a switch 340 coupled in parallel between the charge capacitor 26 and the charge delivery switch 16. A sense signal is tapped off of the series coupled resistors at node 334 and is coupled to the controller 18. The switch 340 is shown in FIG. 2 as being a FET device having a diode coupled across the source and drain of the FET. However, alternative switch designs can be used without departing from the scope of the present invention. The measurement circuit 212 can be used to measure patient impedance during delivery of a therapeutic pulse as described below.

In operation, after determining that defibrillating energy should be delivered to a patient, the charge capacitor 26 is charged to a voltage that is sufficient to deliver an adequate level of defibrillation energy. The charge capacitor is typically charged to approximately 1500 volts or more for delivery of 120-200 Joules of defibrillating energy. The defibrillating energy dose can be delivered in the form of monophasic, biphasic, or multiphasic pulses. As previously mentioned, the embodiment of the charge delivery switch 16 illustrated in FIG. 2 can be controlled by the controller 18 to apply monophasic, biphasic, or multiphasic defibrillation pulses to the electrodes 12A and 12B. For example, to apply a biphasic pulse from the charge capacitor 26 to the electrodes 12A and 12B, the switches 302 and 312 are closed and switches 304 and 310 are opened. This connects the electrode 12A to the charge capacitor 204 and the electrode 12B to a reference potential or ground. Then, to reverse the polarity of the defibrillation pulse, the switches 302 and 312 are opened and the switches 304 and 310 are closed to connect the electrode 12A to reference potential or ground and the electrode 12B to the charge capacitor 204. In a constructed embodiment the switches may be provided by high voltage solid-state switching devices such as IGBTs, as described more fully in U.S. patent application Ser. No. 60/651,432 filed Feb. 8, 2005 by Brink.

FIGS. 3A and 3B illustrate biphasic defibrillation waveforms when applied to patients of low and high patient impedance. Suppose that it is decided to apply a therapeutic dose in the range of 120-200 Joules to defibrillate a patient. For the charge capacitor used and initially charged to a voltage of V₀, the desired dose is delivered in this example when the charge voltage on the capacitor drops to a pulse termination voltage of V_{T}. The tilt can be controlled to apply the desired dose as shown in these examples. For the low impedance patient (FIG. 3A), the voltage slope of the first biphasic pulse 32 is seen to decline rapidly as a large current flow passes through the patient. The first phase of the biphasic pulse ends when the switches 302, 304, 310, 312 are switched and the second phase 34 commences and in this example terminates when the termination voltage level V_{T} is attained and the switches are opened. It is seen that the individual phases 32 and 34 are of short duration as is the overall waveform period T1 due to the low patient impedance.

When the same charge voltage is initially applied to a high impedance patient the voltage slope of the first phase 36 is seen to decline but not as steeply as that for the low impedance patient. The phase is switched and the second phase 38 continues the voltage decline in this example until the termination voltage level V_{T} is reached. It is seen that the duration of each individual phase is longer for the high impedance patient, as is the overall waveform period T2.

As mentioned previously, it is desirable to deliver the energy required to provide a desired probability of defibrillation as efficiently and effectively as possible. Studies have shown that this cannot be done with excessively short waveforms or pulse phases, as very short pulse durations can be too rapid for the membrane response time of the heart myocytes. Thus, excessively short pulses are ineffective for a high probability of defibrillation. Studies have also found that excessively long waveforms or pulse phases, beyond certain durations, do not markedly increase the probability of defibrillation either. Thus, extending a pulse beyond a certain point does not improve the probability of defibrillation and exposes the patient to more energy (and the possibility of tissue damage) without benefit. Accordingly it is desirable to maintain pulse durations in a range which is neither excessively short nor excessively long for both low and high impedance patients.

In an embodiment of the present invention, the patient impedance is measured and the result used by the controller 18 to determine the number of shock phases and/or the individual phase durations. One such embodiment is illustrated by the waveforms of FIGS. 4A, 4B, and 4C. FIG. 4A illustrates a waveform 40 for a patient with a moderate patient impedance. The tilt employed in the delivery of this shock starts from a voltage of an initial value V₀ and declines to a final value of V_{T}. During the period of time T that is required for a waveform of this tilt the waveform contains three alternating phases 42, 44, and 46. In this example, three phase durations are used to deliver a triphasic shock waveform to a patient with a moderate patient impedance.

FIG. 4B shows a waveform 50 of the same tilt for a low impedance patient. For the low impedance patient a shorter period of time T is required to achieve the final voltage value V_{T} and during this time the waveform undergoes two phases 52 and 54. It is seen that the pulse amplitudes steeply decline over the time of each phase. Thus, for a low impedance patient, a biphasic waveform is delivered in this embodiment.

FIG. 4C illustrates a waveform 60 of a shock delivered to a high impedance patient. It is seen that there is very little slope to each phase of the waveform due to the high patient impedance. Hence the time required to deliver the required amount of energy is substantially longer than that of the preceding waveforms. In this embodiment four phases are delivered for the same tilt from V₀ to V_{T}. Thus, for a high impedance patient in this embodiment a four phase waveform is delivered. It is thus seen that the number of phases of each waveform varies in correspondence with the patient impedance.

The ability to vary the number of phases with patient impedance in these examples means that the widths of the phases can be maintained within a narrow range of phase widths. As the patient impedance increases, rather than simply extend the durations of each phase of a waveform with a low number of phases, another phase is added to the waveform and the widths of the phases remains roughly the same. As mentioned above, studies have shown that the probability of defibrillation is not increased significantly beyond a certain point for phases of increasing duration, as characterized by a strength-duration relationship. The probability of defibrillation may be further improved in accordance with the present invention by adding another phase to the waveform in those situations and keeping phase durations within a narrow range. The precise physiological explanation for this is not fully understood. There are numerous theories for this, however. One theory holds that the fibers of the heart muscle, wrapped as they are around the entire body of the heart, are not aligned in a single direction with respect to the electric field of the applied defibrillation pulse. This lack of alignment causes some fibers with their alignment to respond to positive phases and other fibers of different alignment to respond to negative phases of the shock waveform. Furthermore, this theory postulates, the benefit provided to certain fibers by a phase of one polarity is undone to a certain extent by a subsequent pulse phase of the opposite polarity. Keeping the phases short, and of decreasing amplitude and/or duration, however, minimizes the “undoing” of benefit and a prompt return to a phase of the beneficial polarity will continue the benefit of the previous pulse phase of that polarity. Hence, this theory postulates, a shock waveform should keep phases within a range of durations which provide therapeutic benefit to muscle fibers of all alignments but not excessively so as to reverse the benefits of previous pulse phases.

A table of waveform characteristics which is consistent with this latter theory is shown in FIG. 6 with reference to FIG. 5. In this embodiment a substantially constant tilt of V_{T}/V₀ is maintained for each shock waveform produced for delivery of the desired dose. The full period of a waveform is duration T (msec) as shown in FIG. 5. Each pulse phase 72, 74 has a duration of t msec. For waveform durations of from 6 to 12 msec a biphasic waveform is used (two pulse phases) as shown in the table of FIG. 6. Each pulse phase 72, 74 has a duration t of half of the waveform duration as shown by the right-hand column of FIG. 6. In this embodiment pulse phase durations in excess of 6 msec are to be avoided as not improving the probability of defibrillation. Consequently, when the waveform duration T exceeds 12 msec a third pulse phase is added to the waveform and a triphasic waveform (FIG. 4A, for example) is delivered for waveform durations up to 18 msec. At a waveform duration T of 18 msec each pulse phase has a duration t of 6 msec. For waveforms of greater duration T, which would be needed for patients of even higher patient impedances, a fourth pulse phase is added to the waveform (FIG. 4C, for example), causing the individual pulse phases of the waveform to drop to 5 msec for a 20 msec waveform period T. As the individual pulse phases reach their designated limit, another phase of the multiphasic waveform is added and each phase is adjusted to a shorter individual duration. Thus it is seen that the use of the table of FIG. 6 for waveform design is seen to keep the individual phase durations within a range of 3.0 to 6.0 msec as patient impedances increase. Other pulse phase durations may be chosen by individual clinicians. For example some clinicians may favor a pulse phase duration range of 2.5 to 8.0 msec. From a knowledge of the defibrillator capacitance of the charge storage capacitance, the dose desired to be delivered, and the measured patient impedance, the number and duration of the pulse phases can be calculated to maintain a desired range of pulse phase durations.

An embodiment of the present invention can maintain an equal duration for each phase of the shock waveform, or can produce waveforms of varying phase durations. For instance, a waveform of a total duration of 14 msec can be produced by three equal phases of 4.7 msec as shown in the table of FIG. 6, or it can be produced as a triphasic waveform of successively decreasing phase durations of 6 msec, 5 msec, and 4 msec.

Any of a number of different techniques can be used to measure the patient impedance. One technique is to deliver a low level non-therapeutic pulse such as a sinusoid waveform to the patient just prior to delivery of the shock and measure the response of the delivered waveform as described in the aforementioned '751 patent to Morgan et al. The measurement may be done, for example, directly across the patient electrodes 12A 12B. Another technique is to measure the patient impedance across a resistor in series with the patient (as shown if FIG. 2) as the shock waveform is being delivered as for instance during the rise time of the shock voltage as indicated by the circled discontinuity 76 in the leading edge of the initial pulse phase 72 in FIG. 5. The series resistor may then be removed from the circuit by switch 340 to complete the shock waveform. Yet another technique is to calculate the patient impedance from the slope of the voltage waveform. Measurements taken of the voltage level as the waveform declines over the period τ in FIG. 5 can be used to solve for the value Rₚ in the equation V=e^{-τ/RpC}.

What is claimed is:

1. An external defibrillator comprising:
an energy source;
a high voltage circuit coupled to the energy source;
a pair of patient electrodes coupled to the high voltage circuit;
a patient characteristic detection circuit coupled to the patient electrodes; and
a phase duration determination circuit, responsive to the patient characteristic detection circuit and coupled to the high voltage circuit which acts to determine the number of phases of the shock pulse produced by the high voltage circuit.

2. The external defibrillator of claim 1, wherein the patient characteristic detection circuit detects patient impedance.

3. The external defibrillator of claim 2, wherein the phase duration determination circuit further determines the total duration of the shock pulse in response to patient impedance.

4. The external defibrillator of claim 1, wherein the phase duration determination circuit acts to determine the number of phases of a multiphasic waveform of alternating phase polarities.

5. The external defibrillator of claim 4, wherein the phase duration determination circuit further acts to increase the number of phases of a multiphasic waveform to prevent a pulse phase duration from exceeding a given duration.

6. The external defibrillator of claim 5, wherein the phase duration determination circuit further acts to maintain the duration of pulse phases within a desired range of values for patients of different patient impedance.

7. The external defibrillator of claim 4, wherein the phase duration determination circuit further acts to control the tilt of the shock pulse produced by the high voltage circuit.

8. The external defibrillator of claim 7, further comprising an ECG analysis circuit, coupled to the patient electrodes, which acts to determine whether a shock is advised.

9. The external defibrillator of claim 7, wherein the patient characteristic detection circuit acts to measure patient impedance prior to delivery of a shock.

10. The external defibrillator of claim 7, wherein the patient characteristic detection circuit acts to measure patient impedance during delivery of a shock.

11. A method for determining the duration of a multiphasic shock waveform delivered by an external defibrillator comprising:
determining that a shock is advised;
measuring a patient characteristic;
determining the number of phases of a shock waveform from the patient characteristic; and
delivering a shock waveform with the determined number of shock waveform phases.

12. The method of claim 11,
wherein measuring a patient characteristic comprises measuring patient impedance; and
wherein determining the number of phases of a shock waveform further comprises maintaining the durations of the shock waveform phases within a desired range of durations.

13. The method of claim 12, wherein patient impedance is measured prior to delivery of the shock waveform.

14. The method of claim 12, wherein patient impedance is measured during delivery of the shock waveform.

15. The method of claim 12, wherein determining that a shock is advised comprises analyzing an ECG signal.

16. The method of claim 12, further comprising determining the total duration of a shock waveform.

17. The method of claim 16, wherein determining the total duration of a shock waveform comprises determining the tilt of a shock waveform.

18. The method of claim 11, wherein determining the number of phases of a shock waveform further comprises increasing the number of shock waveform phases if the duration of a shock waveform phase in the absence of the increase would exceed a given phase duration.
